(19) United States
(12) Patent Application Publication
(10) Pub. No.: US 2002/0197217 A1
(43) Pub. Date: Dec. 26, 2002

(54) Method and device for human skin tanning with reduced skin damage

(76) Inventors: Sewon Kang, Ann Arbor, MI (US); John J. Voorhees, Ann Arbor, MI (US); Gary J. Fisher, Ypsilanti, MI (US)

Correspondence Address: Bradley N. Ruben, Suite 5A, 463 First Street, Hoboken, NJ, 07030, US

(21) Appl. No.: 10/165,048

(22) Filed: Sep. 18, 2002

Related U.S. Application Data
(60) Provisional application No. 60/296,309, filed on Jun. 6, 2001.

(51) Int. Cl. A61K 7/42; A47B 23/06

(52) U.S. Cl. 424/59; 362/130

(57) ABSTRACT

Tanning can be effected much more safely if radiation limited to the range between 330 nm and 360 nm is used for tanning human skin. Other wavelengths, which may tend to induce MMPs, promote erythema, and/or cause DNA damage.

### BACKGROUND OF THE INVENTION

This invention relates to an apparatus and methods for using that apparatus to affect tanning in human skin with reduced up-regulation of MMPs and with reduced DNA damage.

### THE STATE OF THE ART

It is known generally that UVB radiation causes both sunburn (erythema) and tanning. More recently it has been found that UVA radiation also causes sunburn, although at the earth's surface the amount of UVA radiation is so proportionally smaller than the amount of UVB that the effects of UVA radiation are minimal in comparison. Additionally, it is believed generally that UV radiation causes skin damage, including damage to DNA that can result in neoplasms in the skin. UVC radiation clearly causes significant damage, but it is prevented from reaching the earth's surface by the ozone layer.

In our prior, copending application No. 60/216,244, filed Jul. 6, 2000, the disclosure and figures of which are incorporated herein by reference, we describe that certain wavelengths of sunlight reaching the earth's surface, especially those in the UVB region of 295-325 nm, and those in the UVA region above about 360 nm, are effective at inducing MMPs in human skin. That application provides a composition having UVA and UVB blockers for those wavelengths.

People are generally desirous of having a tanned complexion. In spite of well-publicized research about the “dangers” of tanning, especially related to heightened possibilities of skin cancer and the occurrence of photoaging (e.g., accelerated and enhanced wrinkling and thickening of skin), people everywhere tend to the shorelines and other places where they can tan. Additionally, people go to tanning salons, where UV tanning beds with certain UV tanning wavelengths, and minimal other wavelengths believed to be more dangerous, are used on a once or regular basis to achieve the desired tan. These salons are considered by some to be safer than tanning in sunlight because of the reduced number UV wavelengths. Nevertheless, the wavelengths used in such salons are also the wavelengths that can cause erythema, and also can cause photodamage to the skin.

### SUMMARY AND OBJECTS OF THE INVENTION

In light, and in spite, of the foregoing, as people of lighter skin color generally want to have a darker, tanned complexion, and “sunless tanning” lotions are typically perceived as inadequate to achieve the correct tanning color as one achieves when tanning in sunlight, it would be beneficial to provide a method, and an accompanying device and/or composition, to allow tanning with reduce up-regulation of MMPs and reduced DNA damage in the skin.

We have now discovered that UV wavelengths in the range of from about 330 nm to about 360 nm cause skin darkening in the presence of reduced DNA damage. Accordingly, in one embodiment this invention provides a device that emits UV radiation in the 330-360 nm range only to affect skin tanning. In another embodiment this invention provides a composition comprising UVA and UVB blockers that permit UV radiation in the range of 330-360 nm to penetrate the skin.

### DETAILED DESCRIPTION OF SPECIFIC EMBODIMENTS

All of the experiments described herein were performed on human volunteers, after having given full and informed consent.

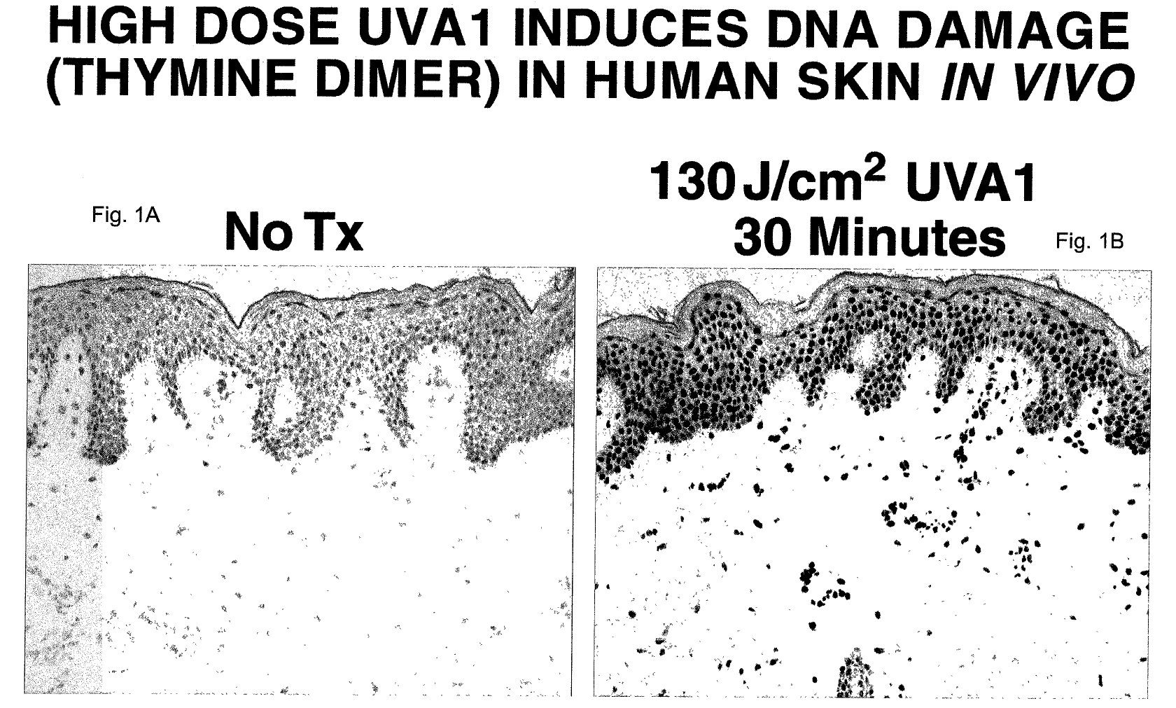

To confirm and qualify the DNA damage that UV radiation causes in human skin, volunteers were exposed to 130 J/cm² from a UVA₁ source for about 30 minutes, and about 30 minutes thereafter their exposed skin was biopsied to determine whether DNA damage occurred. The presence of DNA damaged was based on thymine dimerization, and was detected using a commercially available antibody specific for thymine dimerization. As shown in FIG. 1A, without UVA₁ exposure the skin does not show any staining for the antibody. In FIG. 1B, skin exposed to UVA₁ showed presence of antibody binding to thymine dimer, and hence the presence of DNA damage.

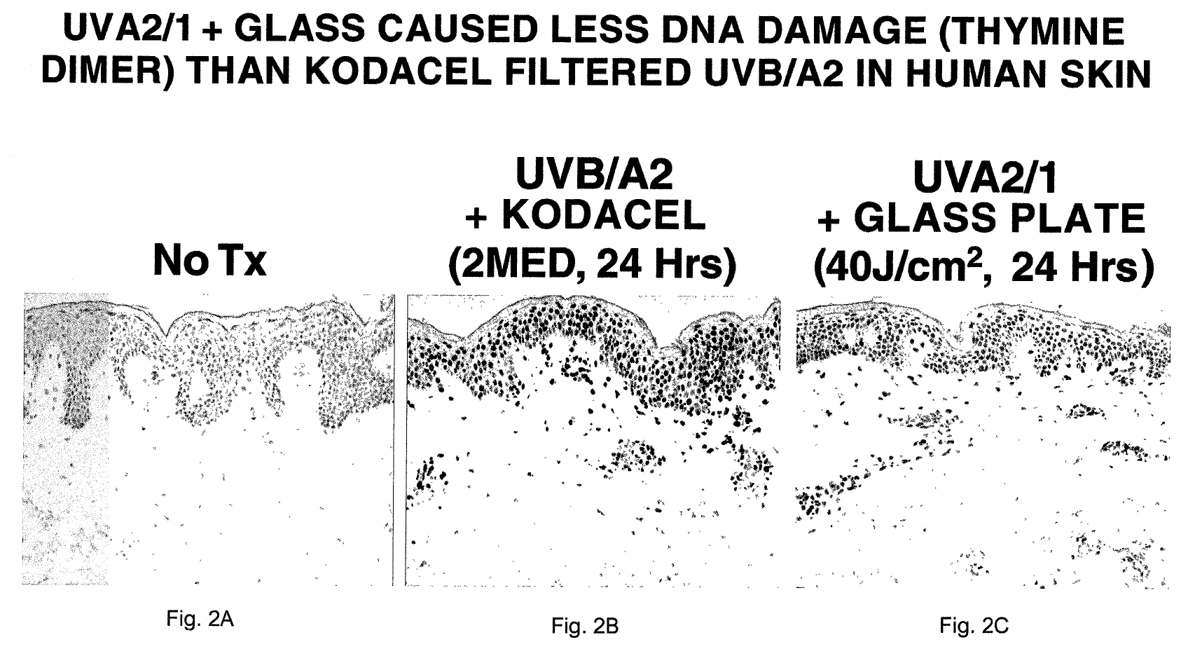

The foregoing experiment was repeated using two different light sources. One was a UVB/A₂ source, that emitted predominantly UVB radiation and having a spectrum that trailed off into the UVA₂ region. The other light source was a UVA_{2/1}. The UVB/A₂ source was filtered with a Kodacel filter, which blocks radiation below about 290 nm. The UVA_{2/1} source was filtered with plain window pane glass. The control, shown in FIG. 2A, does not evidence antibody binding to thymine dimer, presumably because there was no DNA damage without exposure. Volunteers were subjected to an exposure of about 10 min. (about 2 MEDs worth of radiation) to the UVB/A₂ source filtered with a Kodacel filter, and biopsied about 24 hours after exposure. FIG. 2B shows significant antibody binding, suggesting that DNA damage occurs after exposure to UV radiation in the range of 290-325 nm. Volunteers were subjected to an exposure of about 30 min. to the UVA_{2/1} source filtered with a conventional glass plate, equivalent to an exposure of 40 J/cm², with the exposed skin being biopsied about 24 hours after exposure. As shown in FIG. 2C, there is significantly less antibody binding with this latter source filtered with glass.

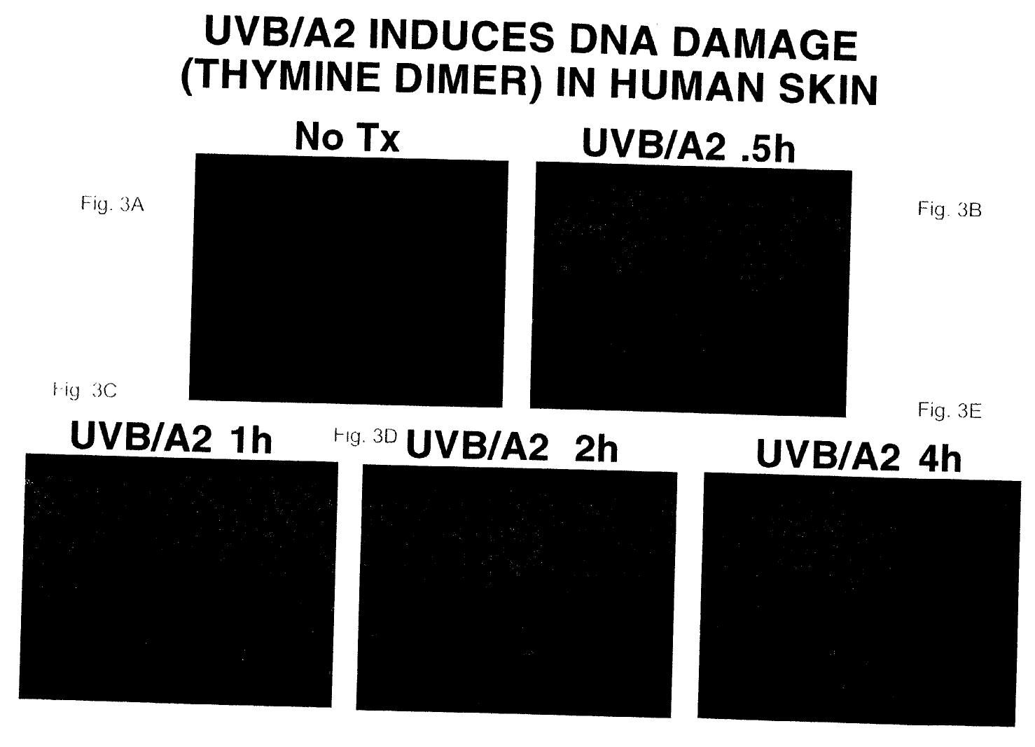

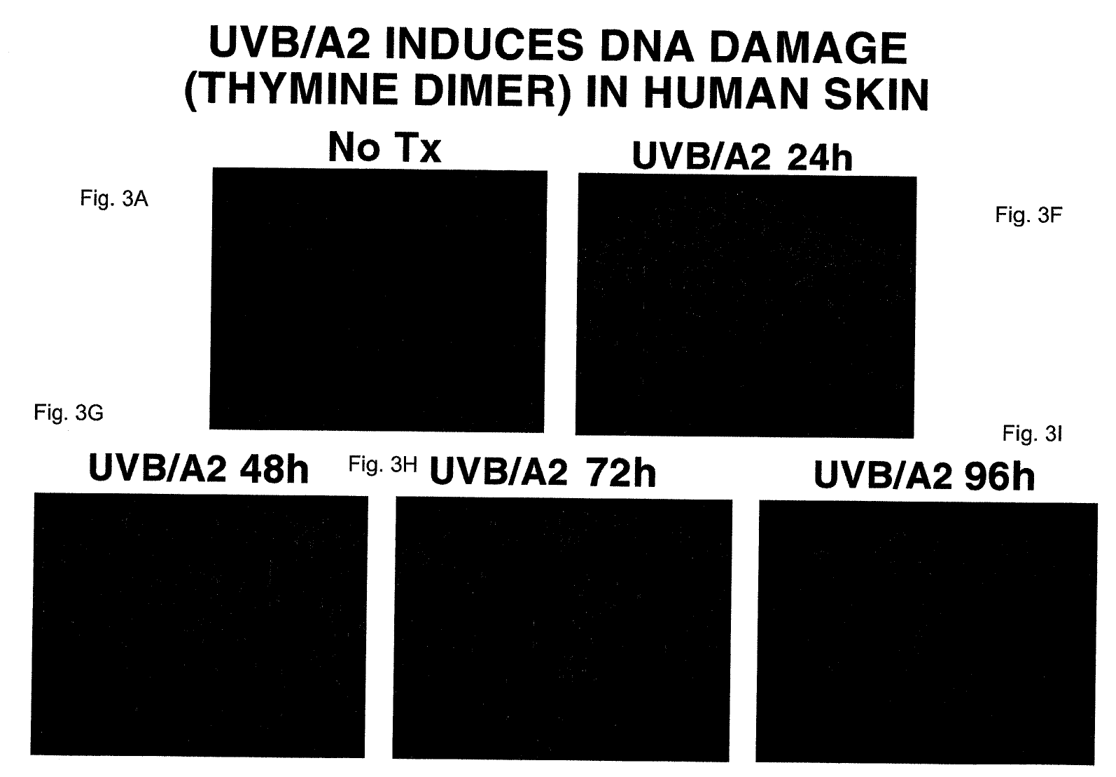

FIGS. 3A-I depict a time course of DNA damage, as determined by antibody-thymine dimer binding in biopsies, over four days; FIG. 3A is the control. Using the above-mentioned UVB/A₂, volunteers' skin was exposed as above (about 2 MEDs of radiation), and then biopsied at various times over the next four days. As seen in FIG. 3B, as little as 30 minutes after exposure to 2 MEDs of UVB/A₂ radiation, DNA damage is present. The other figures show that as biopsies taken as long as 96 hours (four days) after exposure evidence DNA damage.

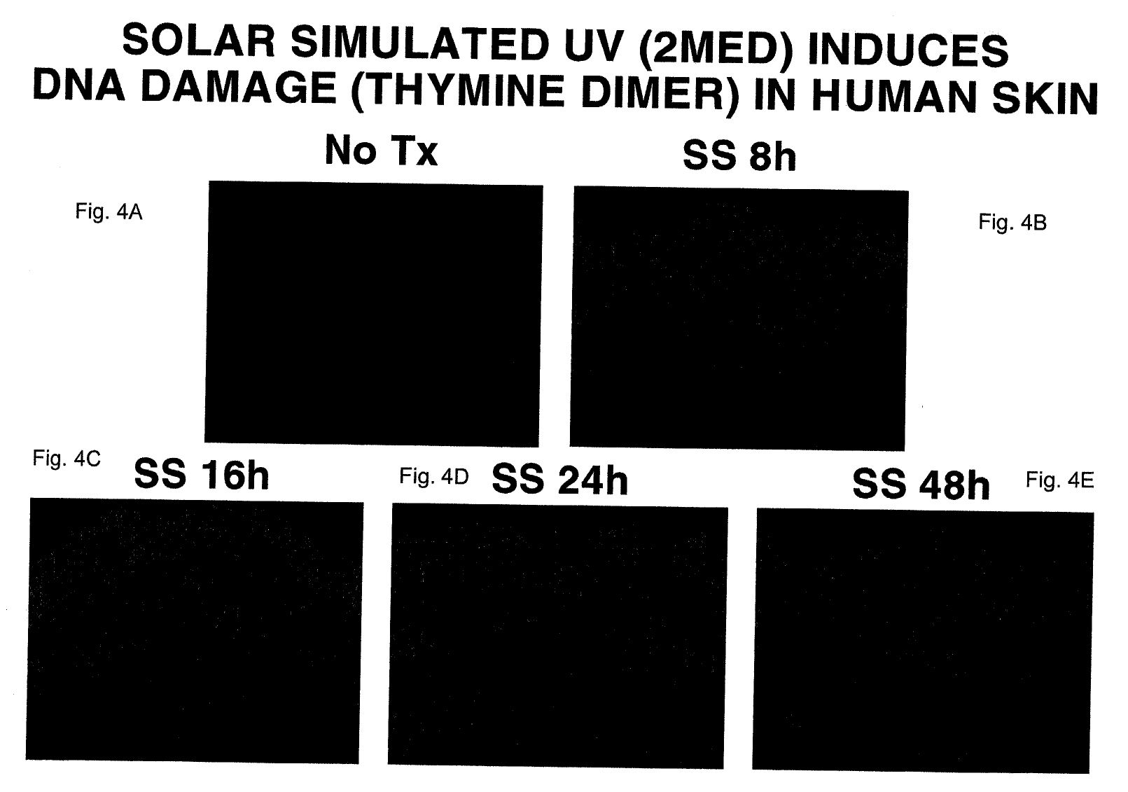

The above-incorporated '244 application describes a solar simulator, which effectively provides a radiation spectrum fairly close to what is actually present at sea level at mid-latitudes. Following the same protocol as used in connection with FIGS. 3A-I, volunteers skin was exposed to 2 MEDs of solar simulated radiation, and biopsied anywhere from eight hours to 48 hours after exposure. Again, as compared with the control shown in FIG. 4A, FIGS. 4B-E show that DNA damage persists for at least a couple of days after an exposure equivalent to a mild sunburn; as above, the volunteers' skin was biopsied after the time shown in the caption to each figure.

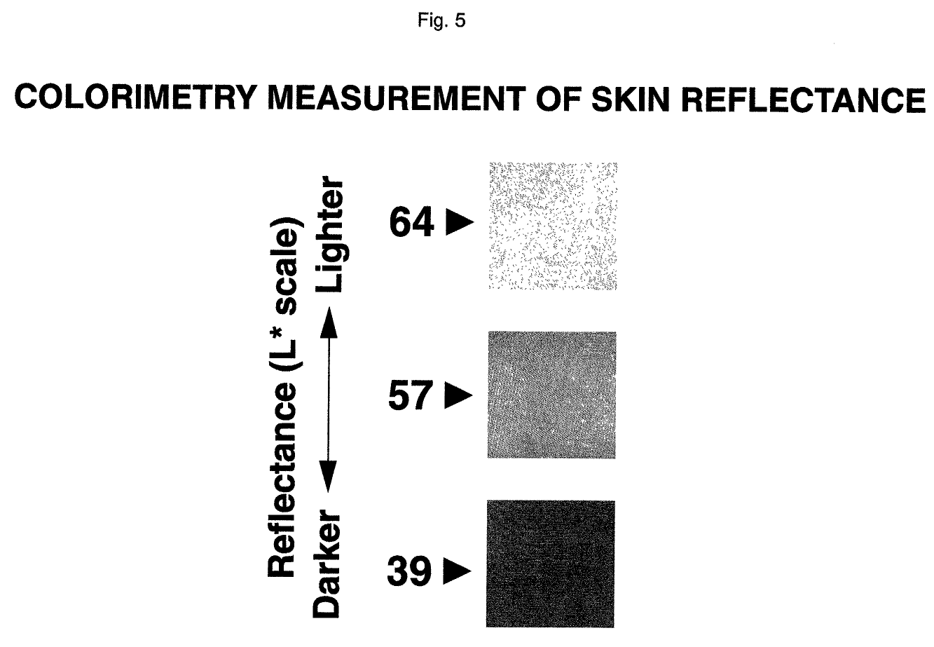

FIG. 5 shows a baseline reading for different human skin types, taken with a Minolta Color Meter model CR-200 chromameter. As shown in the figure, a lower number, indicating lower skin reflectance of light, indicates a darker skin color (L* scale being lower for darker skin).

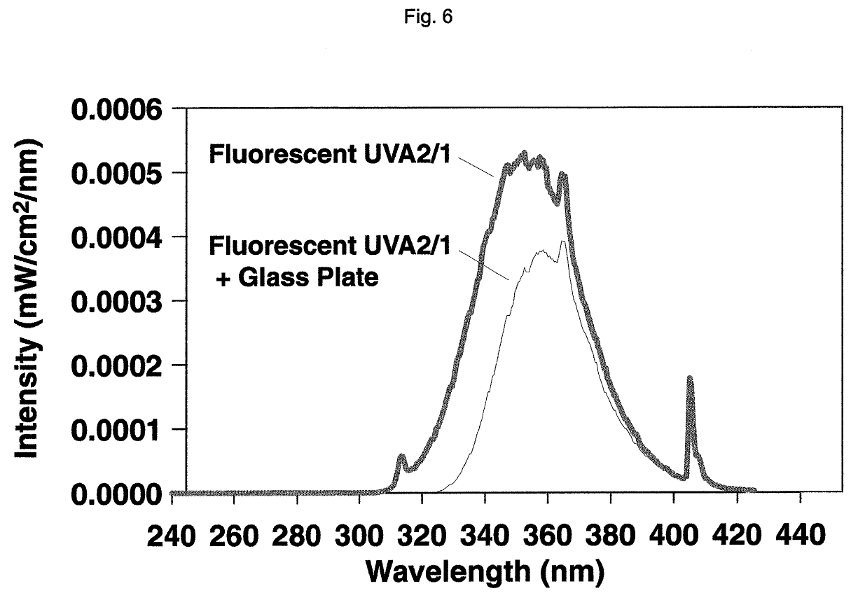

FIG. 6 shows the spectrum for the UVA_{1/2} source used in connection with the results shown in FIG. 2. As seen, the glass plate filters the lower 10-20 nm of the source's UV wavelengths. While also reducing the intensity of the source for wavelengths less than about 360 nm, those less than 330 nm are essentially eliminated.

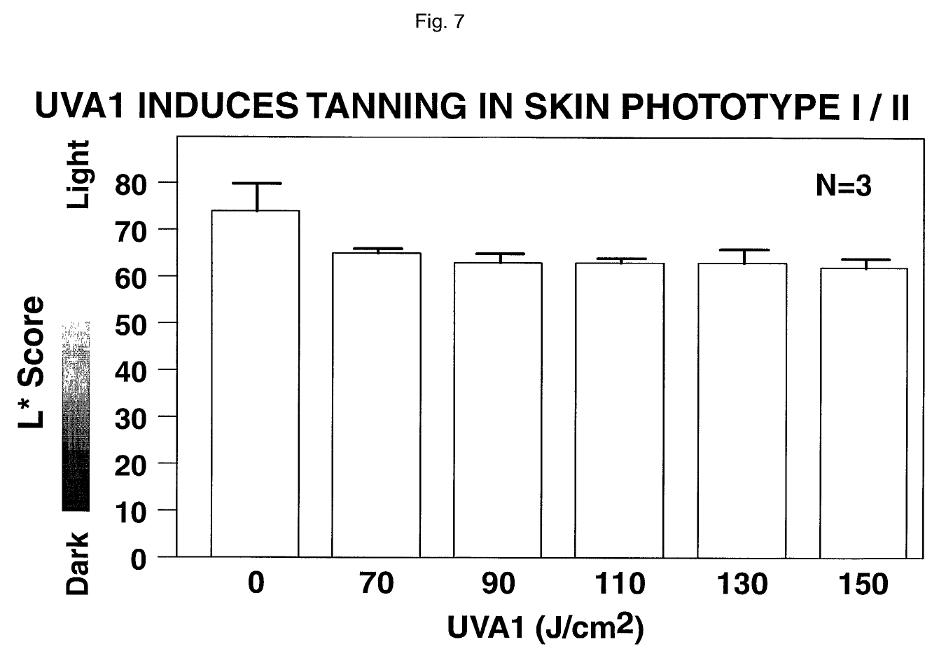

The filtered source having the spectrum in FIG. 6 was used to determine whether such wavelengths promote tanning in human skin. Different patches of skin on three volunteers were exposed to different energies of this filtered UVA_{2/1} source, as shown in FIG. 7. The chromameter reading (per FIG. 5) was taken prior to exposure, and the subjects' readings were average to provide the values shown in FIG. 7. Thereafter, they were exposed to increasing doses of UV radiation and tested again with the chromameter some time later. Although small, the colorimetry reading after exposure to at least 70 J/cm² showed that their skin was darker than the unexposed control, by a statistically significant amount. The dose of radiation did not appear to significantly affect the degree of darkening. Nevertheless, the darkening was generally perceptible to the eye, and when asked whether the darkened area was painful or otherwise uncomfortable when poked, all of the volunteers indicated an absence of pain or discomfort.

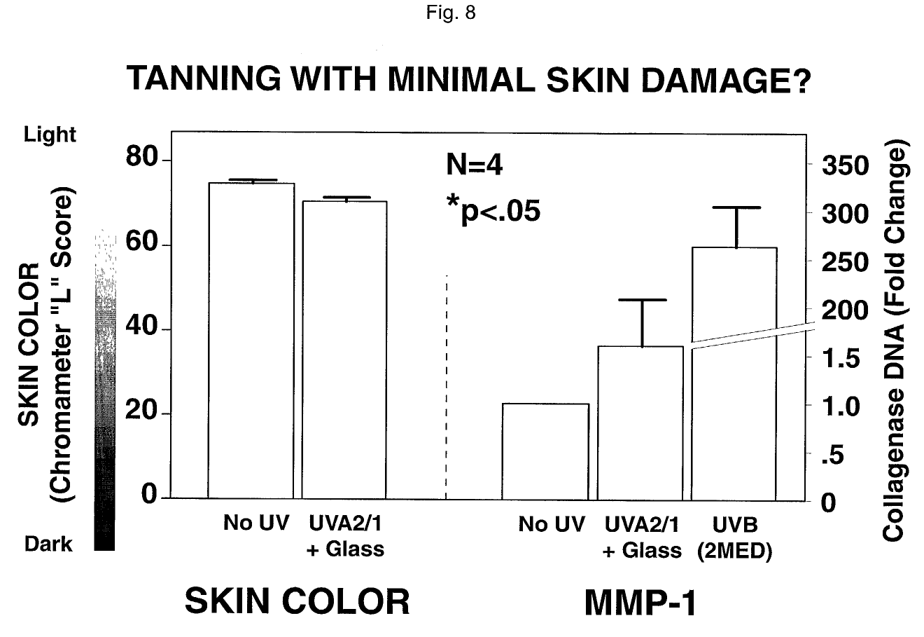

Therefore, is it possible to safely tan? Volunteers were subjected to the UVA_{2/1} source used in the previous experiment. Their skin was biopsied prior to UV exposure to determine a baseline MMP level (for MMPs 1, 3, and 9), and was read with the chromameter to determine a baseline color/reflectivity level. The volunteers' skin was exposed to the filtered UVA_{2/1} source, and shortly thereafter was tested for color change and was biopsied to determine any change in MMP regulation. As shown in the left portion of FIG. 8, the chromameter readings showed a small but statistically significant darkening of the skin. The right portion of FIG. 8 shows that the MMP-1 levels changed minimally after exposure to the filtered UVA_{2/1} source, but increased significantly after exposure to 2 MEDs of UVB. Although not shown, levels of MMP-3 and MMP-9 also increased from baseline after exposure to the UVB source, but exposure to the filtered UVA_{2/1} source did not increase them any more than MMP-1 was increased.

In summary, the above experiments show that sources of UVA and/or UVB can induce DNA damage, that filtering a UVA_{2/1} source can virtually eliminated indicators of DNA damage, and that the filtered UVA_{2/1} source can darken human skin. Further, in connection with our co-pending '244 application, the wavelengths of the UVA_{2/1} source used in this application are not those described in the '244 application as being particular detrimental because they are effective for enhancing MMP activity.

Thus, an apparatus for providing a “safe” tan comprises a source of UV radiation limited to wavelengths between about 330 nm and about 360 nm.

A composition for “safe” tanning is a sunscreen having blockers for UV radiation, at least in the 295-325 nm and >360 nm wavelength ranges, and permitting UV radiation between about 330 nm and about 360 nm to penetrate through to the skin.

With respect to the apparatus, a method for “safe” tanning comprises exposing human skin to the source of UV radiation for a plurality of sessions. While a single exposure may only lower the L* scale value by a few points, repeated exposures would be expected to further lower the value. Accordingly, after a number of weeks, with exposure daily or every other day, one would be expected to have a reasonable tan.

With respect to the composition, the method comprises applying the composition to areas of the skin to be exposed to the sun, and then going out into the sun. Of course, the composition could be used in conventional tanning salon booths, and with the inventive apparatus as a further measure of safety.

Accordingly, it can be seen that a combination of proper UV wavelengths from a radiation source (including at least 330-360 nm sufficient to cause tanning) and a sunscreen can be used in combination to achieve the desired effect. For example, a UV source not emitting above about 360 nm (or 350 nm, or 340 nm, so long as there is illumination in the 330-360 nm range) but emitting UVB light can be used in combination with a UVB blocker to provide, via a combination of the UV source (which can be filtered to provide illumination in the 330-360 nm range) and a sunscreen (UVB blocker), a safe tanning environment.

A suitable apparatus can include UVA lamps such as Q-Panel UVA-351, available from Q-Panel Lab Products, Cleveland, Ohio, in combination with known filters, such as UV34 2.5, SF12 2, or WG360 2.5, which filter in the UVA1 range.

The foregoing description is meant to be illustrative and not limiting. Various changes, modifications, and additions may become apparent to the skilled artisan upon a perusal of this specification, and such are meant to be within the scope and spirit of the invention as defined by the claims.

What is claimed is:

1. An apparatus for providing a “safe” tan that provides minimal MMP induction, minimal erythema, and minimal DNA damage, comprising a source of UV radiation providing UV wavelengths only between about 330 nm and about 360 nm and essentially excluding UV wavelengths that induce MMPs or cause erythema.

2. The apparatus of claim 1, wherein the source comprises a broader spectrum UV source and a filter to limit the wavelengths to between about 330 nm and about 360 nm.

3. A composition for “safe” tanning that provides minimal MMP induction, minimal erythema, and minimal DNA damage, comprising: a sunscreen having blockers for UV radiation, at least in the UVB range of 295-325 nm and in the UVA range of >360 nm wavelength ranges, and permitting UV radiation between about 330 nm and about 360 nm to penetrate through to the skin to effect tanning.

4. A method for “safe” tanning that provides minimal MMP induction, minimal erythema, and minimal DNA damage, comprising treating the skin of a human being with the apparatus of claim 1.

5. A method for safe tanning that provides minimal MMP induction, minimal erythema, and minimal DNA damage, comprising: applying to human skin the composition of claim 3, and exposing the skin so treated to a UV source having radiation composition for “safe” tanning is a sunscreen having blockers for UV radiation between about 330 nm and about 360 nm.

6. The method of claim 5, wherein the UV source is an apparatus.

7. The method of claim 5, wherein the UV source is the sun.

8. A method for safe tanning that provides minimal MMP induction, minimal erythema, and minimal DNA damage, comprising illuminating human skin with a source of UVA radiation in the 330 nm to 360 nm wavelength, and minimizing other UV wavelenghts from illuminating the skin by a combination of applying to human skin at least one of a UVB blocker for UVB range of 295 nm to 325 nm, a UVA blocker for the UVA range of >360 nm, and one or more filters between the radiation source and the human skin.

9. The method of claim 8, wherein the method is a combination of a UVB blocker and filtering.

10. The method of claim 8, wherein the method is a combination of a UVA blocker and filtering.

11. The method of claim 8, wherein the method is a combination of a UVA blocker, a UVB blocker, and filtering.f
